(19) United States
(12) Patent Application Publication
(10) Pub. No.: US 2018/0264206 A1
(43) Pub. Date: Sep. 20, 2018

(54) ATTACHMENT FOR OR ON A DEVICE FOR INJECTING A FLUID INTO OR UNDER THE SKIN

(71) Applicant: Hahn-Schickard-Gesellschaff fur angewandte Forschung e.V., Villingen-Schwenningen (DE)

(72) Inventors: Markus CLEMENZ, Villingen-Schwenningen (DE); Michael KEGEL, Tennenbronn (DE)

(21) Appl. No.: 15/572,827

(22) PCT Filed: May 12, 2016
(86) PCT No.: PCT/EP2016/060692

§ 371 Date: Jun. 1, 2018

(30) Foreign Application Priority Data
May 13, 2015 (DE) 10 2015 107 613.6

(51) Int. Cl. A61M 5/46; A61M 5/34; A61K 38/48; A61K 9/00

(52) U.S. Cl. CPC A61M 5/46; A61M 5/345; A61M 2205/584; A61K 9/0019; A61K 38/4893

(57) ABSTRACT

An attachment (1, 1a) for or on a device (100) for injecting a fluid into or under the skin (200), which is for example suitable for injecting botulinum toxin into facial muscles of a human patient, comprises: a proximal part (2, 2a) which is couplable or is connected to the device (100) and which bears a cannula (3). A distal part (4, 4a) surrounds the cannula (3) at least in places and is rotationally mobile relative to the proximal part (2, 2a) and vice versa, such that an extent (d₃) by which the cannula projects out from the distal part (2, 2a) is modifiable. A discrete number of stopping positions is provided with regard to the rotational movement, which correspond to a different extent by which the cannula (3) projects out from the distal part (2, 2a).

### FIELD OF THE INVENTION

The invention relates to an attachment for or on a device for injecting a fluid into or under the skin, for example botulinum toxin into a human patient.

### BACKGROUND OF THE INVENTION

Administering botox (botulinum toxin) may be difficult because the attending physician must very skillfully manually set the puncture depth of the syringe at a puncture angle of between 45° and 90°. In the field of dermal access devices in which a puncture is made subcutaneously with a puncture angle of less than 25°, DE 10 2010 001 506 A1 discloses locking a cannula for injection of the fluid to be administered in two positions, the cannula being further extended in the first position and the second position being reached by withdrawing the cannula. WO 2009/107945 A2, U.S. Pat. No. 8,858,498 B2, WO 2013/121307 A1, DE 72 42 473 U and WO 2012/029082 A1 disclose the provision of multipart attachments for syringes, a distal part being mobile relative to a proximal part and an extent by which a cannula projects out from the distal part being modifiable by the motion.

In the case of the device according to WO 2009/107945 A2, a distal part is provided with an internal thread and a proximal part of the attachment is provided with an external thread at its distal end. The distal part is screwed onto the external thread of the proximal part. A scale is provided on the proximal part in front of the external thread and, depending on the extent to which the scale is covered, different maximum numerical values are visible. One drawback of this arrangement is the poor retention of the distal part if the cannula is intended to protrude only slightly and a maximum number on the scale is readable. A further drawback is that no discrete positions are settable and the setting can only be checked visually and not by tactile and acoustic means. A further drawback is that the numerical values on the scale do not indicate the depth of injection, but instead the shortening of the inserted cannula. A further drawback are potential sources of error which arise from the possibility of selecting different cannulas for use with the instrument.

In the attachment of U.S. Pat. No. 8,858,498 B2, two or more stable positions are provided which can be stably occupied by the distal part relative to the proximal part. WO 2013/121307 A1 shows a plurality of stable catch positions of the distal part. In the case of the puncture depth setting from DE 72 42 473 U, it is known to provide successive sawtooth structures on an axis by means of which the attachment can be stably locked relative to a syringe. WO 2012/029082 A1 likewise reveals a two-part attachment for a syringe, a needle of the syringe penetrating both attachment parts when the syringe rests against the proximal, inner attachment part. Puncture depth can be set by rotating the proximal attachment part relative to the distal attachment part. Low stability of the arrangement is a drawback here too.

The concept of displacing the entire syringe relative to an attachment is furthermore known. In the case of a puncture depth setting from EP 2 523 707 B1, for example, a syringe for insulin travels on a carriage relative to a housing. The method proceeds by means of a gear unit which comprises a large toothed ring outside the carriage and a small toothed ring on a rotatable cylinder as part of the carriage. US 2001/0047151 A1 provides a screw nut on a syringe attachment, the nut simultaneously providing a seat for an apron on the needle end of the syringe. DE 200 135 79 U1 furthermore discloses a refillable dispenser and injector, in which a puncture depth of a cannula can be fixed by an adjusting nut. In order to control the puncture depth of a needle, it is known from WO 98/50094 A1 to mount the needle on an inner body which is provided with an external thread which is rotatable in an internal thread of an outer body. Rotation proceeds by actuating an adjusting ring. The needle is extended out from one end and simultaneously extended out of the fluid reservoir for the fluid to be injected. The fluid reservoir may optionally be pushed back by manual actuation. The drawback here is that the entire device is very complex and susceptible to failure.

### Problem Addressed by the Invention

The problem addressed by the present invention is that of providing reliable technical assistance when introducing botox into and under the skin of a human patient.

### Inventive Solution

The problem addressed by the invention is solved according to a first aspect of the invention by an attachment for or on a device for injecting a fluid into or under the skin, for example botulinum toxin into a human patient, the attachment comprising a proximal part which is couplable or connected to the device and which bears a cannula, the attachment furthermore comprising a distal part which surrounds the cannula at least in places and the proximal part and the distal part being rotationally mobile relative to one another in such a manner that an extent by which the cannula projects out from the distal part is modifiable. Rotational mobility implies that, in the event of rotational motion of the proximal part relative to the distal part or vice versa, a discrete number of stopping positions may be achieved. A stopping position is a position in which motion out of the stopping position is restricted in at least one direction by a resistance. The resistance may preferably be overcome by application of force. In one embodiment of the invention, motion is restricted in a direction which effects retraction of the cannula into the distal part. In one embodiment of the invention, motion is restricted in a direction which effects extension of the cannula into the distal part. Motion is preferably restricted both in a direction which effects retraction of the cannula into the distal part and in a direction which effects extension of the cannula into the distal part. The resistance is preferably achieved by a catch mechanism. There are preferably at least two, particularly preferably at least three, more preferably at least five, and at most highly preferably at least ten such stopping positions, with between one and twenty, for example at least three, four, five or ten stopping positions being for example passed through per millimeter of extension length of the cannula out of the distal part. As an alternative to unilaterally acting limit stops, a catch mechanism is provided which provides a plurality of catch positions, which correspond to a different extent by which the cannula projects out from the distal part. For the purposes of the present application, a catch position may be provided by an interlocking connection, but may alternatively also be achieved by a non-interlocking connection, for example a variation in frictional resistance is achievable by a local modification of the material or the surface roughness. The preferred catch means comprises one or more groove(s) and at least one resilient catch member which engages in the groove(s).

The invention enables an attending physician to cause the cannula to project out of the distal part by a defined length. This length corresponds to the subsequent puncture depth, when the cannula is inserted until the distal part comes into contact with the skin, for example with a suitably configured end face. Provision of the stopping positions provides the attending physician with haptic or tactile and optionally also acoustic feedback regarding the extension length of the cannula. Moreover, further adjustment may also be possible for a cannula which has already been inserted into the human patient by the next or a further catch position being occupied by now rotating the proximal part relative to the distal part. Extension by rotation in the opposite direction may also be possible.

### Preferred Configuration of the Invention

Advantageous further developments which may be used individually or in combination with one another are the subject matter of the dependent claims relating to the first aspect of the invention.

It is preferably provided in relation to the attachment that the catch positions are successively passed through on rotation of the distal part in the same direction of rotation and the extent by which the cannula projects out here either continuously further increases or continuously further reduces. Such a solution is mechanically very simple to implement.

The preferred attachment comprises a toothed ring of axially extending teeth with axially extending grooves located therebetween and furthermore comprises a resilient catch member which, in a catch position, engages in one of the grooves. The toothed ring may thus be rotated when the proximal or the distal part is rotated. The catch positions are then naturally passed through during rotation. If the resilient catch member, as is preferably provided, takes the form of a metal strip, it is not only very simple to provide but also ensure stable locking.

If the toothed ring is provided on the proximal part and is arranged closer to the cannula than a thread which permits rotational motion of the distal part relative to the proximal part, it is then possible to ensure that the distal part is more stably retained on the proximal part than if the thread is provided further forwards, i.e. further distally. In addition, locking is stabler if it is provided in the vicinity of the cannula and thus of the patient.

If the toothed ring is provided on an inner side of the distal part, it is possible to ensure that a larger number of catch positions can be occupied with a given outer circumference of the attachment than if the toothed ring is provided on the outer side of the proximal part.

The preferred attachment according to the invention comprises a spiral groove on the outside of the proximal part, in which groove the distal part engages. In this manner it is possible to ensure that the distal part entirely surrounds the proximal part and, for assembly, can be simply put in place. In the case of the proximal part being provided with a toothed ring which is closer to the cannula than it is to the spiral groove, it is further preferred for the distal part and the proximal part each to be of circular cross-section and for the toothed ring to have a maximum radial extent which is smaller than the radial distance of a base of the spiral groove. In other words, the proximal part is less thick at its distal end than at its end facing the device for injecting fluid. A fine, thin tip accordingly creates a precision tool which permits very fine puncturing and does not cover a wide area around the puncture site during puncture, such that the fluid for injection can be administered in a highly targeted and precise manner.

The spiral groove may advantageously be constructed such that an information-bearing portion is arranged between its various portions (i.e. raised between the spiral grooves), in particular a portion bearing a number, a letter, a symbol, a scale mark, a gray value from a gray scale or a color from a color scale or a combination of one or more of these above-stated elements. The spiral groove is thus extended over a greater axial length, where a greater axial range can be passed through by the distal part with fewer rotations, so the cannula may be exposed and lengthened with relatively few rotations or conversely shortened in terms of the extent by which it projects out. This embodiment may be optimized by suitably providing the catch mechanism with a plurality or even a multiplicity of catch positions. The respective puncture depth or the extent by which the cannula projects out from the distal part is preferably readable as the item of information.

As an alternative to the spiral groove on the outside of the proximal part, the preferred attachment according to the invention comprises an internal thread in an interior cavity of the proximal part, in which thread a stem of distal part engages. In this case, the outer surface of the proximal part can be more freely designed.

As an alternative to the toothed ring of axially extending teeth, it is possible to provide a plurality of teeth with interspaces (grooves), a resilient catch member engaging in an interspace (groove) when it occupies a catch position, it being resiliently mobile in an axial direction. Resilient loading in an axial direction ensures a greater degree of stability during injection.

In a third alternative, dispensing with a separate toothed ring in the region of a spiral groove provided on the outside of the proximal part, in which groove the distal part engages, a plurality of catch indentations are provided, these possibly being concave and round or taking the form of polygonal cutouts or indentations. This embodiment has the advantage of great compactness.

A second aspect of the invention provides an attachment for or on a device for injecting a fluid into or under the skin, for example botulinum toxin into a human patient, the attachment comprising a proximal part which is couplable or connected to the device and which bears a cannula, the proximal part comprising in one portion a spiral groove on its outer side. A distal part which surrounds the cannula at least in places engages in the spiral groove, the proximal part and the distal part being rotationally mobile relative to one another in such a manner that the extent by which the cannula projects out from the distal part is modifiable. Axially successive portions of the spiral groove are spaced part by raised portions of the proximal part that bear an item of information which is indicated with the assistance of numbers, letters, symbols, scale marks and/or a gray or color scale or a combination of one or more of these above-stated elements. The respective puncture depth or the extent by which the cannula projects out from the distal part is preferably readable as the item of information. In other words it is here provided that the spiral groove travels a relatively large axial distance with only a slight rotation. In order to make optimal use of space, the scale, which in particular represents the information as to how far the cannula projects out, is provided between the portions of the spiral groove.

In particular, it is preferably provided that the spiral groove extends over an axial portion of between 20% and 60% of the total axial extent of a cannula-bearing body of the proximal part. In other words, a very large proportion of the proximal part is used to provide the spiral groove, whereby the puncture depth (the extent by which the cannula projects out) can be varied to a very great extent in relation to the total size of the attachment.

A third aspect of the invention provides an attachment for or on a device for injecting a fluid into or under the skin, in particular botulinum toxin into a human patient, the attachment comprising a proximal part which is couplable or connected to the device, which comprises a cannula and in one portion comprises a spiral groove on its outer side. A distal part which surrounds the cannula at least in places engages in the spiral groove. The distal part and the proximal part are rotationally mobile relative to one another in such a manner that the extent by which the cannula projects out from the distal part is modifiable. The spiral groove extends over an axial portion of between 20% and 60% of the total axial extent of a cannula-bearing body of the proximal part. A very large proportion of the proximal part is thus here used for the spiral groove such that, in relation total size of the attachment, the extent by which the cannula projects out can be varied to a very great extent.

According to all three aspects of the attachment, a means is preferably provided on the distal part, with the assistance of which the distal part can be fixed on the proximal part in one position, the position here preferably being one in which the cannula does not project out from the distal part. Protection from needlestick injury prior to injection when setting up the device for injection or also after retraction of the cannula and in the case of a changeover of the device for injection is thus provided. It is preferably possible with this means, for instance in the form of a detent lever, to achieve still more stable retention in position than with the assistance of the catch mechanism.

A fourth aspect of the invention provides an attachment for or on a device for injecting a fluid into or under the skin, for example botulinum toxin into a human patient, the attachment comprising a proximal part which is couplable or connected to the device and which bears a cannula. A distal part surrounds the cannula at least in places and is translationally mobile relative to the proximal part, such that an extent by which the cannula projects out from the proximal part is modifiable. An adjusting screw engages in a thread on the proximal or distal part and, on the outside of the other part, comprises a grip, for example with ribbing, for holding. When such an adjusting screw is provided for displacing the distal part relative to the proximal part, the screw itself ensures stable retention.

In all four stated aspects of the attachment according to the invention, the distal part preferably comprises at least one portion made of a resiliently elastic material, such that a user can compress the distal part to an extent such that the proximal part is grippable. Handling is facilitated as a result.

In one embodiment, the distal part comprises an end face which is flat. Alternatively, such an end face which is curved may be present, for example overall convex or overall concave. A flat or curved end face ensures that the distal part can be set down stably on the skin of a human patient, it in particular acting as a suitable endpoint of the injection movement. An attending physician can more readily achieve the injection angle of between 45° and 90° which is preferred for botox injection if the end face is suitably formed.

As a further alternative, the distal (free) end of the distal part may be of pointed construction, preferably tapering conically, so facilitating visual monitoring during puncture.

In a preferred embodiment in all four stated aspects, the distal part is transparent at its distal end at least in places (for example only over part of its circumference) or in its entirety. As a result, the attending physician can visually monitor injection of the cannula into the skin and subsequently into the facial muscle.

The distal part is preferably provided in all four aspects of the invention with a transparent window, optionally with a magnifying glass, an opening or also an electrically operated display device. A scale located underneath, in particular on the proximal part, can be read through the window, magnifying glass or opening. The electrically operated display may likewise provide information about the puncture depth (or the extent by which the cannula projects out from the distal part).

The attachment may be provided in various ways.

The attachment may comprise on its proximal part a connector for coupling to the device for injecting the fluid, for example in the form of a Luer Lock®, a Luer Slip®, a cone for coupling a syringe or a tube connection. As a result, the attachment can be provided separately and flexibly coupled to different types of devices for injecting a fluid.

Alternatively, the attachment may be integral with the device for injecting a fluid, such that the attending physician need not fit it him/herself and the device for injecting a fluid can be compactly stored with the attachment.

Further alternatively, the attachment may be integrated in an ultrasound head for or of an ultrasound device.

Finally, the attachment may also be provided in or on a dispensing/regulating attachment for a device for injecting a fluid, i.e. two attachment units may be provided jointly.

### BRIEF DESCRIPTION OF THE DRAWINGS

The invention is explained in more detail below with reference to drawings, in which:

FIG. 1: shows an attachment according to a first exemplary embodiment in a perspective, partially cutaway view, the cannula not protruding from the attachment;

FIG. 2: shows the attachment from FIG. 1 in a position in which the cannula is projecting out by a distance d₃;

FIG. 3: shows an exploded view of the attachment from FIG. 1;

FIG. 4: shows the attachment from FIG. 1, in a section along line IV-IV, in perspective view;

FIG. 5: shows section IV-IV from FIG. 1;

FIG. 6: shows the attachment from FIG. 1 with a syringe mounted thereon, the attachment resting at an angle of 90° on a plane diagrammatically representing a patient's skin;

FIG. 7: shows the attachment from FIG. 6, but rotated by an angle of less than 45°;

FIG. 8: shows a perspective detail of the proximal part with a detent lever which belongs to the distal part according to a first embodiment in a locking position and

FIG. 9: shows the representation from FIG. 8 in unlocked position;

FIG. 10: shows a perspective detail of the proximal part with a detent lever which belongs the distal part according to a second embodiment in a locking position and

FIG. 11: shows the representation from FIG. 10 in unlocked position;

FIG. 12: shows an attachment according to a second exemplary embodiment in a perspective, partially cutaway view, the cannula not protruding from the attachment;

FIG. 13: shows an exploded representation of the attachment from FIG. 12;

FIG. 14: shows an attachment according to a third exemplary embodiment in a perspective, partially cutaway view, the cannula not protruding from the attachment;

FIG. 15: shows the attachment from FIG. 14 in the longitudinal section XV-XV;

FIG. 16: shows a dispensing/regulating attachment which likewise embodies the invention in one aspect.

### DETAILED DESCRIPTION OF THE INVENTION ON THE BASIS OF EXEMPLARY EMBODIMENTS

The intention is to facilitate the administration of botulinum toxin (botox) with the assistance of a syringe (displaceable piston in a fluid reservoir), specifically the intention is in particular firstly to facilitate precise setting of the depth of the injection and to prevent excessively shallow or excessively deep puncture. The intention is furthermore to facilitate making a plurality of rapid, repetitive injections one after the other to an identical depth.

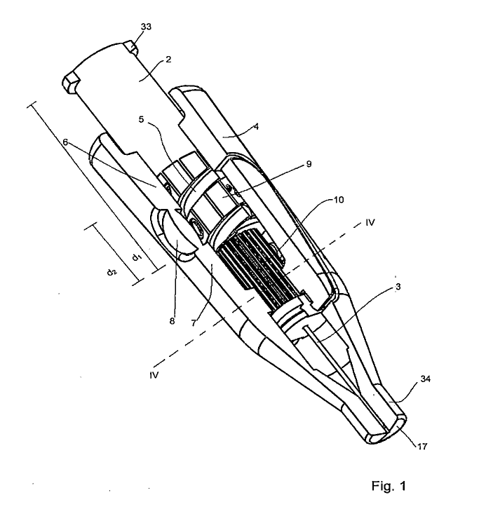

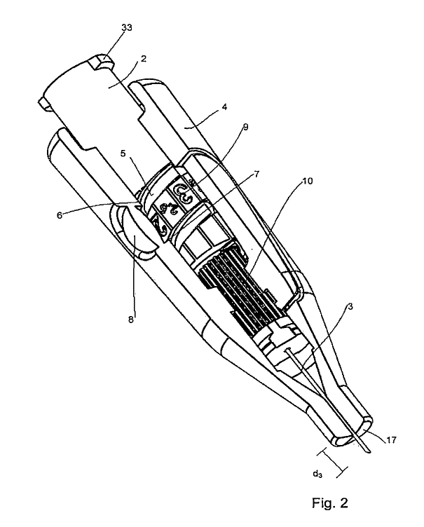

FIG. 1 shows a first exemplary embodiment of an attachment 1 according to the invention with a proximal part 2, which bears a cannula 3, and with a distal part 4. The cannula 3 is guided axially through the entire proximal part axial and protrudes out from its distal end. The distal part 4 surrounds the proximal part 2 and in particular also the cannula 3 at least in places, i.e. over at least part of the circumference and/or part of the length measured in an axial direction. The proximal part 2 comprises a spiral groove 5, in which two guide tips or pins 6 and 7 of the distal part 4 engage. Between the guide tips 6 and 7 is located a magnifying lens 8, through which is visible a raised part 9, which is located between two portions of the spiral groove 5 and which bears a scale, in the present case in the form of numerical values. The spiral groove 5 extends over a total length d₂ of a main body of the proximal part 2 with the axial extent d₁ and, as the guide tips 6 and 7 slide in the spiral groove 5, enables extension of the cannula 3 by a length d₃, see FIG. 2. The scale is configured such that an indication of the respective puncture depth can be read as an item of information through the magnifying lens 8.

The ratio of d₂ to d₁ amounts to 40%.

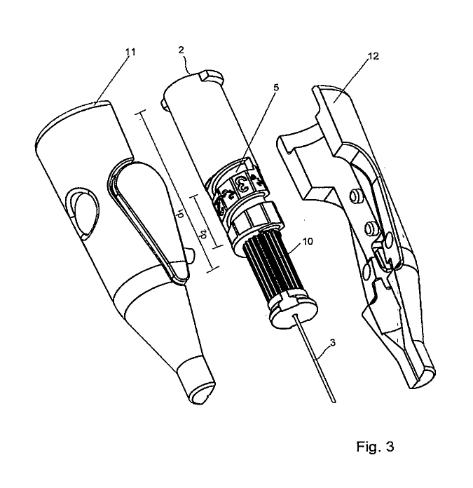

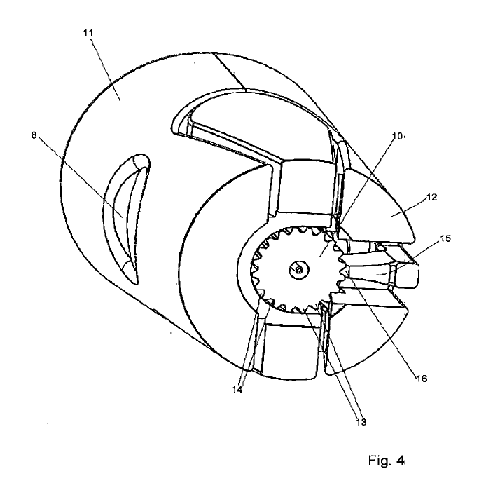

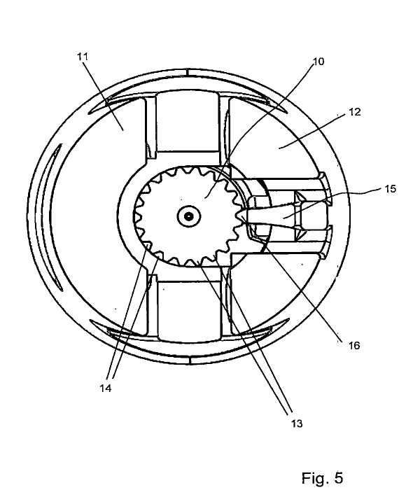

The main body, which bears the spiral groove 5 and in relation to which the axial extent d₁ is defined, is adjoined by a cylindrical portion 10 which differs from the main body and bears a toothed ring with teeth 13, between which are located interspaces in the form axial grooves 14, see FIGS. 3 to 5. A strip 15, the tip 16 of which engages in the interspaces 14 between the teeth 13, is arranged on the distal part 4. If the distal part 4 is rotated relative to the proximal part 2, the metal strip 15 is resiliently bent until its tip 16 leaves one interspace 14 and snaps into the next, adjacent interspace 14. In this manner, the distal part 4 can be held stable relative to the proximal part 2 while a puncture depth of the cannula 3, i.e. the extent d₃ by which it projects out, can nevertheless be gradually increased or reduced. In particular, the situation can be adapted to the patient to be injected.

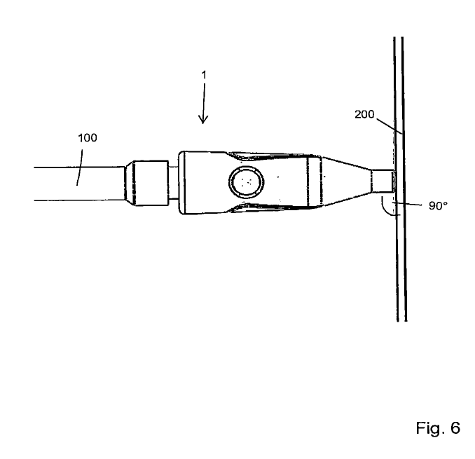

FIG. 6 shows the attachment 10 with a syringe body 100 coupled thereto as a device for injecting fluid into or under the skin, in particular of botulinum toxin into facial muscles of a human patient. FIG. 6 shows the skin diagrammatically as a plane 200. The syringe is fastened to the attachment 1 with the assistance of a Luer Lock® 33 (see FIGS. 1 and 2) provided on the proximal part 2.

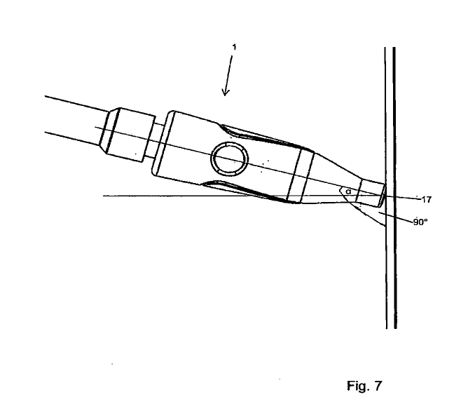

The end face 17, which is partially visible in FIGS. 1 and 2, may be flat or concavely or convexly curved, a relatively small angle of less than 15° also being provided in the case of a curvature at the edge relative to the region around the exit point of the cannula 3. As is visible from FIGS. 6 and 7, the syringe 100 with the attachment 1 can be stably set down at an angle of 90° or deviate slightly from 90° by an angle α which amounts to at most 45°.

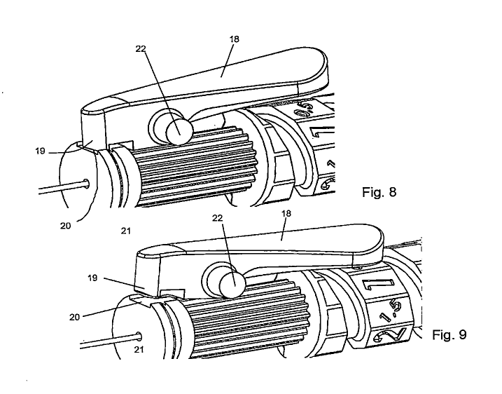

In particular when the situation according to FIG. 1 prevails, i.e. the cannula 3 is not projecting out of the distal part 4, it is desired for the distal part 4 to lock stably relative to the proximal part 2, so offering protection from needlestick injury during insertion of the syringe and after withdrawal of the cannula. FIG. 8 shows in this connection a portion of the proximal part with a detent lever 18, which belongs to the distal part, which is moreover not shown in FIG. 8. A catch projection 19 which can engage in a cutout 20 of a plate 21 can be released by actuating the detent lever 18. By actuating the detent lever 18, during which the latter rotates about a shaft 22, the release position shown in FIG. 9 can be achieved and the distal part 4 rotated relative to the proximal part 2 in order to extend the cannula.

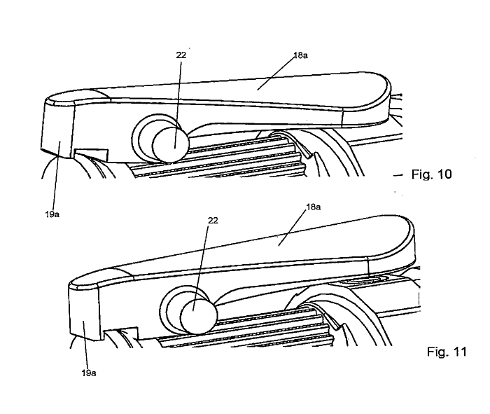

While in the case of FIGS. 8 and 9, the catch projection 19 prevents rotational motion of the distal part relative to the proximal part, in the case of the embodiments according to FIGS. 10 and 11 axial motion of the distal part relative to the proximal part is prevented, so providing protection from needlestick injury during insertion of the syringe and after withdrawal of the cannula.

FIG. 12 shows a second exemplary embodiment of an attachment 1a according to the invention in which the proximal part 2a has cutouts 24 which are triangular in the region of its groove 5a, such that a series of teeth 23 are obtained, into which the guide tip 6 can snap. In this case, the groove 5a itself provides the catch means. The cylindrical body 10 with the toothed ring is no longer necessary and a spring element 25 is provided instead, which rests against an annular seat 26 of the distal part 4a. FIG. 13 shows this embodiment in exploded view.

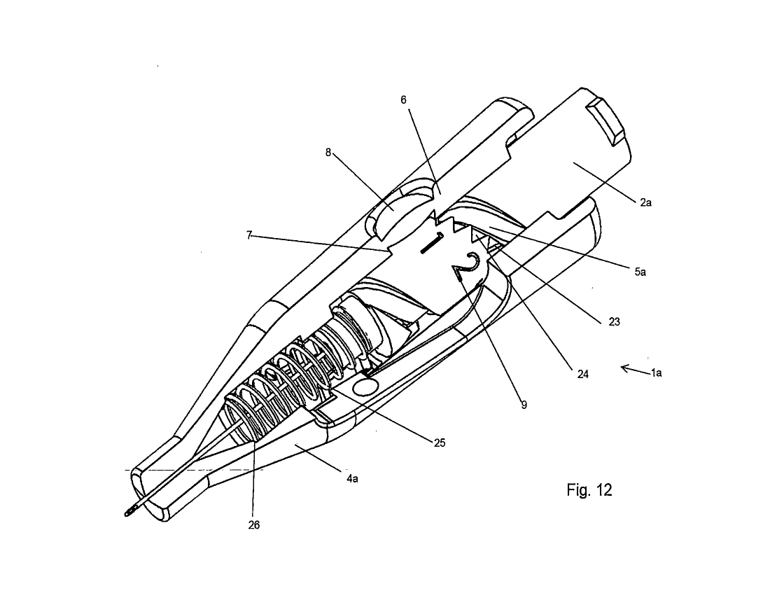

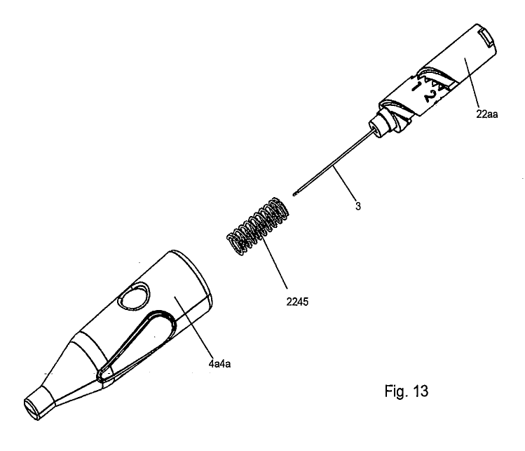

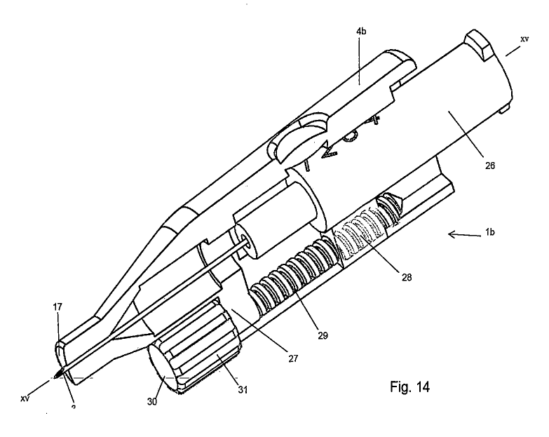

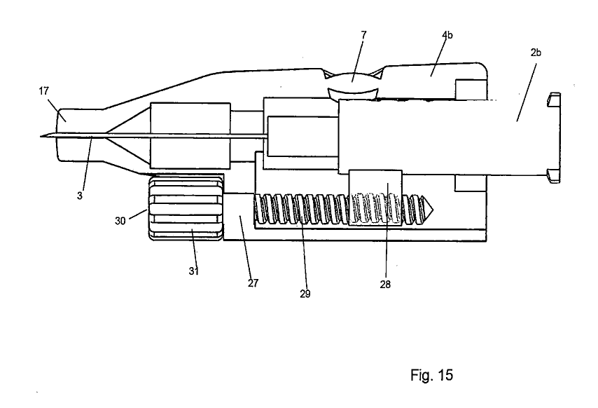

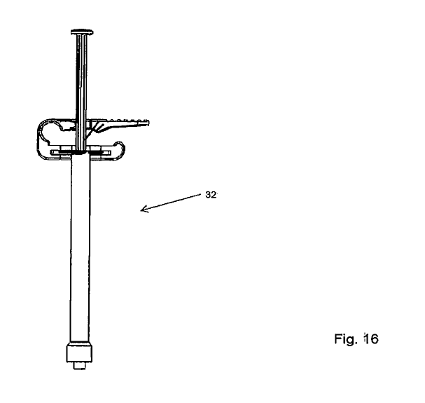

FIG. 14 shows a third exemplary embodiment of an attachment 1b according to the invention in which a lug 28 is formed on the proximal part 2b and a lug 27 on the distal part 4b. An adjusting screw 29 is guided through the two lugs 27 and 28 and in particular engages in an internal thread, indicated by broken lines in FIG. 14, of lug 28. An internal thread may optionally also be provided in lug 27. A head 30 of the adjusting screw 29 is provided with ribbing 31 to facilitate handling: an operator can rotate the top 30 and so cause the adjusting screw to displace the proximal part 2b axially relative to the distal part 4b. This translational motion results in the cannula 3 emerging from the end face 17 of the distal part 4b.

The attachment 1 can be connected externally to a syringe via the Luer Lock® 33 or may instead also be integrated into an ultrasound head for observing the site at which the syringe is to be introduced. In the case of FIG. 16, a dispensing/regulating attachment for a syringe is combined with the attachment 1 or 1b according to one of the embodiments of the invention.

In all the embodiments, the distal part 4 or 4a, 4b may consist of very soft plastic, whereby compression of the distal part and direct retention of the proximal part 2, 2a, 2b is made possible. The cylindrical outer surface 34 is preferably transparent in the region of the end face 17, such that the end of the cannula 3 can also be seen during the injection procedure. The cannula 3 is suitably beveled, as is for example visible in FIG. 14, in order to permit precise placement of the cannula into or under the skin or facial skin of a human patient and then onwards into a facial muscle, into the subcutaneous tissue or another organ or tissue.

The features disclosed in the above description, the claims and the drawings may be of significance for implementation of the invention in its various embodiments either individually or in any desired combination.

1. An attachment for or on a device for injecting a fluid into or under the skin, the attachment comprising: a proximal part which is couplable or connected to the device and which bears a cannula, a distal part which surrounds the cannula at least in places and the proximal part and the distal part being rotationally mobile relative to one another in such a manner that an extent (d₃) by which the cannula projects out from the distal part is modifiable, wherein a discrete number of stopping positions is provided with regard to the rotational motion, which correspond to a different extent by which the cannula projects out from the distal part.

2. The attachment as claimed in claim 1, wherein a catch mechanism provides a plurality of catch positions.

3. The attachment as claimed in claim 1, wherein the catch positions are successively passed through on rotation of the distal part in the same direction of rotation and the extent (d₃) by which the cannula projects out here either continuously further increases or continuously further reduces.

4. The attachment as claimed in claim 3, wherein a toothed ring of axially extending teeth with axially extending grooves is located therebetween, and with a resilient catch member which, in a catch position, engages in one of the grooves, the resilient catch member taking the form of a strip.

5. The attachment as claimed in claim 4, wherein the toothed ring is provided on the proximal part and is arranged closer to the cannula than a thread which permits rotational motion of the distal part relative to the proximal part.

6. The attachment as claimed in claim 4, wherein the toothed ring is provided on an inner side of the distal part.

7. The attachment as claimed in claim 1, wherein a spiral groove is provided on the outside of the proximal part in which groove the distal part engages.

8. The attachment as claimed in claim 5, wherein the distal part and the proximal part each have a circular cross-section, in that the toothed ring has a maximum radial extent which is smaller than the radial distance of a base of the spiral groove from the center point of the circular cross-section.

9. The attachment as claimed in claim 7, wherein a portion which bears information is provided between different portions of the spiral groove the portion having at least one element from a number, a letter, a symbol, a scale mark, a gray value on a gray scale or a color on a color scale, or having a combination of one or more of these elements.

10. The attachment as claimed in claim 1, wherein an internal thread is provided in a cavity of the proximal part which is open towards the distal end, in which thread a stem of the distal part engages.

11. The attachment as claimed in claim 2, wherein a plurality of teeth with interspaces are provided, in which interspaces a resilient catch member engages, the catch member being resiliently mobile in an axial direction when it occupies a catch position.

12. The attachment as claimed in claim 2, wherein a spiral groove is provided on the outside of the proximal part, in which groove the distal part engages, catch indentations being provided on or in the spiral groove.

13. An attachment for or on a device for injecting a fluid into or under the skin, the attachment comprising: a proximal part which is couplable or connected to the device, which bears a cannula and in one portion comprises a spiral groove on an outer side, in which groove a distal part engages, the distal part surrounding the cannula at least in places and the proximal part and the distal part being rotationally mobile relative to one another in such a manner that the extent (d₃) by which the cannula projects out from the distal part is modifiable, wherein axially successive portions of the spiral groove are spaced apart by raised portions of the proximal part that bear an item of information which is indicated with the assistance of numbers, letters, symbols, scale marks and/or a gray or color scale.

14. The attachment as claimed in claim 13, wherein the spiral groove extends over an axial portion of between 20% and 60% of the total axial extent of a cannula-bearing main body of the proximal part.

15. An attachment for or on a device for injecting a fluid into or under the skin, the attachment comprising: a proximal part which is couplable or connected to the device, which bears a cannula and in one portion comprises a spiral groove on an outer side, in which groove a distal part engages, the distal part surrounding the cannula at least in places and the proximal part and the distal part being rotationally mobile relative to one another in such a manner that the extent (d₃) by which the cannula projects out from the distal part is modifiable, wherein the spiral groove extends over an axial portion of between 20% and 60% of the total axial extent of a cannula-bearing body of the proximal part.

16. The attachment as claimed in claim 1, wherein a means is provided on the distal part, with the assistance of which the distal part can be fixed on the proximal part in one position, in which the cannula does not project at all out of the distal part, in order to provide protection from needlestick injury prior to injection when setting up the device for injection or after retraction of the cannula or in the case of a changeover of the device for injection.

17. An attachment for or on a device for injecting a fluid into or under the skin, the attachment comprising: a proximal part which is couplable or connected to the device and which bears a cannula, and a distal part which surrounds the cannula at least in places and is translationally mobile relative to the proximal part, such that an extent (d₃) by which the cannula projects out from the proximal part is modifiable, wherein an adjusting screw engages in a thread on the proximal part or on the distal part and a grip for holding.

18. The attachment as claimed in claim 1, wherein the distal part comprises at least one portion of a resiliently elastic material, such that a user can compress the distal part to an extent such that the proximal part is grippable.

19. The attachment as claimed in claim 1, wherein the distal part comprises an end face which is flat or curved, preferably overall convex or overall concave.

20. The attachment as claimed in claim 1, wherein the distal part tapers conically at its distal end.

21. (canceled)

22. (canceled)

23. (canceled)

24. (canceled)

25. (canceled)

26. (canceled)
